(19) United States
(12) Patent Application Publication
(10) Pub. No.: US 2010/0009911 A1
(43) Pub. Date: Jan. 14, 2010

(54) Osteogenic synergic composition

(75) Inventor: Gerard Soula, Meyzieu (FR)

Correspondence Address: OLIFF & BERRIDGE, PLC, P.O. BOX 320850, ALEXANDRIA, VA, 22320-4850, US

(73) Assignee: ADOCIA, Lyon (FR)

(21) Appl. No.: 12/457,095

(22) Filed: Jun. 1, 2009

Related U.S. Application Data
(60) Provisional application No. 61/129,011, filed on May 30, 2008.
(60) Provisional application No. 61/129,618, filed on Jul. 8, 2008.

(30) Foreign Application Priority Data
Jul. 7, 2008 (FR) 08 54617

(51) Int. Cl. A61K 38/18; A61P 19/08

(52) U.S. Cl. 514/12

(57) ABSTRACT

The invention relates to an osteogenic synergic composition comprising at least one osteogenic growth factor, and at least one growth factor having a chemoattractant and angiogenic capacity. It also relates to the method for the preparation thereof and to the use thereof for the preparation and production of pharmaceutical products for use in bone reconstruction and regeneration, in the form of topical compositions, for example implants, pastes or gels.

The present invention relates to the field of osteogenic formulations, and more particularly formulations of osteogenic proteins belonging to the family of Bone Morphogenetic Proteins, BMPs.

Bone Morphogenetic Proteins (BMPs) are growth factors involved in the mechanisms of osteoinduction. BMPs, also known as Osteogenic Proteins (OPs), were initially characterized by Urist in 1965 (Urist MR. Science 1965; 150, 893). These proteins, isolated from cortical bone, have the ability to induce bone formation in a large number of animals (Urist MR. Science 1965; 150, 893).

The present invention relates to the combination, with a Bone Morphogenic Protein, BMP, of another growth factor having chemoattractant and angiogenic capacities, such as Platelet Derived Growth Factor, PDGF, for promoting bone formation.

This combination leads to synergy between the two growth factors and makes it possible to create a bone mass that is greater than that obtained with just one of these growth factors, including at doses lower than those commonly used.

It is known by those skilled in the art that BMPs make it possible to differentiate stem cells into osteoblasts capable of generating bone. Two therapeutic products are sold for osteogenic applications:
Infuse (Medtronic), which is a formulation of BMP-2 for the fusion of lumbar vertebrae and of nonunion fractures of the tibia.
OP1 (Stryker), a BMP-7-based product, also for the fusion of lumbar vertebrae.

A Japanese university team, the studies of which were published in the British Journal of Oral and Maxillofacial Surgery, 2003, 41, 173-178, has established that, in rats, PDGF-BB and BMP-2 are secreted during the healing process for a mandibular fracture.

It is accepted that PDGF-BB is not a cell differentiation factor, which is confirmed by the fact that PDGF-BB does not make it possible to create bone formations on an ectopic site.

However, a PDGF-BB-based product has been developed by BioMimetic for bone regeneration in the case of periodontal disease.

In application WO 2007/092622, reference is made to the combination of PDGF with other growth factors, without this reference being supported by any result or example.

Another Japanese team has published, in the Journal of Bone and Mineral Research, 2002, 17, 2, 257-265, that PDGF-BB has a role in the bone remodelling process, but that it is responsible:
for an increase in activity of osteoclasts, which are responsible for bone degradation;
for an inhibition of osteoblasts responsible for bone formation.

None of these studies has taught that a synergic effect from the point of view of osteosynthesis can be obtained for a formulation combining a growth factor of each of the abovementioned two families.

It is to the applicant's credit to have obtained, surprisingly, a formulation based on BMP and on PDGF which is found to synergically promote osteosynthesis.

The invention thus relates to an osteogenic synergic composition comprising at least one osteogenic growth factor, and at least one growth factor having a chemoattractant and angiogenic capacity.

The term “osteogenic growth factor” or “BMP”, alone or in combination is intended to mean, a BMP selected from the group of therapeutically active BMPs (Bone Morphogenetic Proteins).

More particularly, the osteogenic proteins are selected from the group constituted of BMP-2 (dibotermin-alfa), BMP-4, BMP-7 (eptotermin-alfa), BMP-14 and GDF-5, alone or in combination.

The BMPs used are recombinant human BMPs, obtained according to the techniques known to those skilled in the art or purchased from suppliers such as, for example, the company Research Diagnostic Inc. (USA).

The term “chemoattractant and angiogenic growth factors” is intended to mean a protein selected from the group constituted of PDGF, VEGF or FGF, alone or in combination.

The invention thus relates to an osteogenic synergic composition comprising at least one osteogenic growth factor, and at least one growth factor having a chemoattractant and angiogenic capacity, PDGF.

In one embodiment, the invention relates to a synergic composition comprising at least one osteogenic protein selected from the group constituted of BMP-2 (dibotermin-alfa), BMP-4, BMP-7 (eptotermin-alfa), BMP-14 and GDF-5, alone or in combination, and at least one growth factor having a chemoattractant and angiogenic capacity, PDGF.

In one embodiment, the invention relates to a composition comprising at least BMP-2 and PDGF-BB.

In one embodiment, the invention relates to a composition comprising at least BMP-7 and PDGF-BB.

The invention thus relates to an osteogenic synergic composition comprising at least one osteogenic growth factor, and at least one growth factor having a chemoattractant and angiogenic capacity, VEGF.

In one embodiment, the invention relates to a synergic composition comprising at least one osteogenic protein selected from the group constituted of BMP-2 (dibotermin-alfa), BMP-4, BMP-7 (eptotermin-alfa), BMP-14 and GDF-5, alone or in combination, and at least one growth factor having a chemoattractant and angiogenic capacity, VEGF.

The invention thus relates to an osteogenic synergic composition comprising at least one osteogenic growth factor, and at least one growth factor having a chemoattractant and angiogenic capacity, FGF.

In one embodiment, the invention relates to a synergic composition comprising at least one osteogenic protein selected from the group constituted of BMP-2 (dibotermin-alfa), BMP-4, BMP-7 (eptotermin-alfa), BMP-14 and GDF-5, alone or in combination, and at least one growth factor having a chemoattractant and angiogenic capacity, FGF.

In another embodiment, the bone formation is all the more promoted if the growth factors are formulated with an amphiphilic polymer capable of forming complexes with said growth factors.

The invention thus relates to a synergic composition as defined above, characterized in that it further comprises an anionic polysaccharide selected from the group constituted of anionic polysaccharides functionalized with hydrophobic derivatives selected from derivatives of dextrans bearing hydrophobic substituents such as tryptophan and tryptophan derivatives.

According to the invention, the functionalized dextran can correspond to the following general formulae:

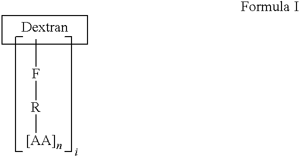

R being a chain containing between 1 and 18 carbons, which is optionally branched and/or unsaturated, which comprises one or more heteroatoms, such as O, N and/or S, and which has at least one acid function;
F being either an ester, a thioester, an amide, a carbonate, a carbamate, an ether, a thioether, or an amine;
AA being a hydrophobic L- or D-amino acid residue produced from the coupling between the amine of the amino acid and an acid borne by the R group, said hydrophobic amino acid being selected from tryptophan derivatives, such as tryptophan, tryptophanol, tryptophanamide, 2-indole ethylamine, and alkali-metal cation salts thereof;

i is the molar fraction of substituent F—R—[AA]ₙ per glycosidic unit and is between 0.1 and 2;

n is the molar fraction of M-substituted Rgroups and is between 0.05 and 1;

when R is not substituted with AA, then the acid(s) of the R group is (are) cation carboxylates, the cation preferably being a cation of an alkali metal such as Na, K,

said dextran being amphiphilic at neutral pH.

In one embodiment, the alkali-metal cation is Na⁺.

In one embodiment, F is either an ester, a carbonate, a carbamate or an ether.

In one embodiment, the polysaccharide according to the invention is a carboxymethyl dextran (DMC) of formula IV:

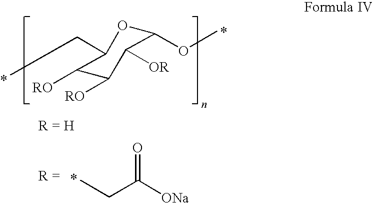

or the corresponding acid.

In one embodiment, the polysaccharide according to the invention is a monosuccinic ester of dextran or succinic acid dextran (DSA) of formula V:

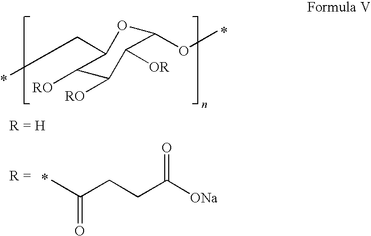

or the corresponding acid.

In one embodiment, the polysaccharide according to the invention is characterized in that the R group is selected from the following groups:

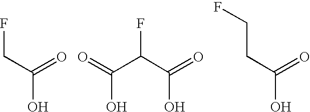

or the alkali-metal cation salts thereof.

In one embodiment, the dextran according to the invention is characterized in that the tryptophan derivatives are selected from the tryptophan esters of formula II:

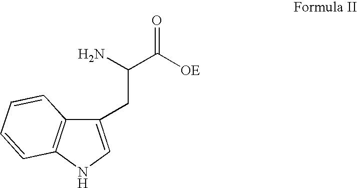

E being a group that may be:
a linear or branched C₁ to C₈ alkyl,
a linear or branched C₆ to C₂₀ alkylaryl or arylalkyl.

In one embodiment, the dextran according to the invention is a carboxymethyl dextran modified with the ethyl ester of tryptophan of formula VI:

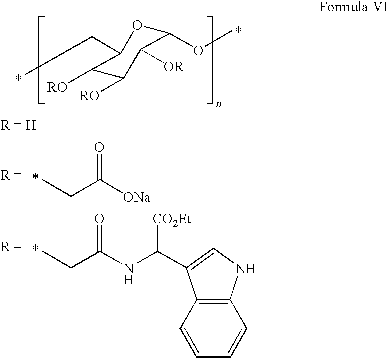

In one embodiment, the dextran according to the invention is a monosuccinic ester of dextran or succinic acid dextran (DSA) modified with the ethyl ester of tryptophan of formula VII:

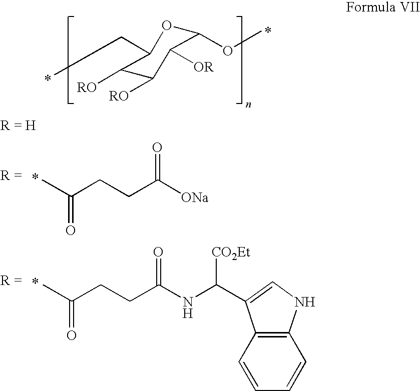

In one embodiment, the dextran according to the invention is characterized in that the hydrophobic amino acid is phenylalanine or the alcohol, amide or decarboxylated derivatives thereof.

In one embodiment, the dextran according to the invention is characterized in that the phenylalanine derivatives are selected from the esters of this amino acid of formula III:

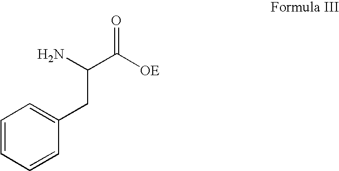

E being defined as above.

The dextran may have a degree of polymerization m of between 10 and 10 000.

In one embodiment, it has a degree of polymerization m of between 10 and 1000.

In another embodiment, it has a degree of polymerization m of between 10 and 500.

In another embodiment, the bone formation is also promoted in the presence of soluble divalent- or multivalent-cation salts.

The invention relates to a composition characterized in that it also comprises a divalent cation selected from the group constituted of calcium, magnesium or zinc cations.

It relates to a composition characterized in that the soluble divalent-cation salt is a calcium salt, the counterion of which is selected from the chloride, the D-gluconate, the oxalate, the hydroxide, the formate, the D-saccharate, the phosphate, the carbonate, the acetate, the sulphate, the L-ascorbate, the L-tartrate, the L-lactate, the glutamate or the aspartate.

In one embodiment, the soluble divalent-cation salt is calcium chloride.

In one embodiment, the invention relates to a composition according to any one of the preceding claims, characterized in that it further comprises multivalent cations selected from the group constituted of iron cations, aluminium cations, and cationic polymers selected from polylysine, spermine, protamine and fibrin, alone or in combination.

The invention also relates to a composition as defined above, characterized in that it further comprises an organic matrix selected from matrices based on sterilized, preferably crosslinked, purified natural collagen.

The invention also relates to the use of synergic compositions according to the invention, for the preparation and production of pharmaceutical products for use in bone reconstruction or regeneration, in the form of topical compositions, for example in the form of implants.

The synergic compositions according to the invention are prepared by solubilization of the growth factors in a solution buffered at physiological pH.

In one embodiment, a polymer solution is added to the growth factor solution.

In one embodiment, the solution is added to a matrix selected from matrices based on sterilized, preferably crosslinked, purified natural collagen.

In one implant embodiment, the formulations according to the invention are lyophilized before use.

The invention thus relates to a formulation as defined above, characterized in that it is in the form of a lyophilisate.

The term “lyophilisate” is intended to mean the product or the composition resulting from a lyophilization procedure.

Lyophilization is a water sublimation technique enabling dehydration of the composition. This technique is commonly used for the storage and stabilization of protein.

The rehydration of a lyophilisate is very rapid and enables a ready-to-use formulation to be easily obtained, it being possible for said formulation to be rehydrated before implantation, or implanted in its dehydrated form, the rehydration then taking place, after implantation, through the contact with the biological fluids.

The osteogenic compositions according to the invention are used by implantation, for example, for filling bone defects, for performing vertebral fusions or maxillofacial reconstructions, or for treating an absence of fracture consolidation (pseudarthrosis).

In these various therapeutic uses, the size of the matrix and the total amount of growth factors depend on the volume of the site to be filled.

In one embodiment, for a vertebral implant, the doses of growth factors will be between 0.05 mg and 8 mg, preferably between 0.1 mg and 4 mg, more preferably between 0.1 mg and 2 mg, whereas the doses commonly accepted in the literature are between 8 and 12 mg of BMP-2.

As regards the uses in maxillofacial reconstruction or in the treatment of pseudarthrosis, for example, the doses administered will be of the order of about 10 μg.

In one embodiment, the cation solutions have concentrations of between 0.01 and 1M, preferably between 0.05 and 0.2M.

In one embodiment, the solutions of anionic polysaccharide have concentrations of between 1 mg/ml and 2 mg/ml, preferably between 5 and 100 mg/ml, more preferably between 10 and 50 mg/ml.

The invention also relates to the use of the composition according to the invention as a bone implant.

In one embodiment, said composition may be used in combination with a prosthetic device of the vertebral prosthesis or vertebral fusion cage type.

The invention also relates to the therapeutic and surgical methods using said composition in bone reconstruction.

The invention also relates to the method for preparing the compositions according to the invention, which comprises at least the following steps:
a) providing an organic matrix,
b) impregnating said matrix with a solution comprising the two growth factors and/or the amphiphilic anionic polysaccharide/growth factor complex(es) and/or the amphiphilic anionic polysaccharide,
c) adding a solution of a soluble salt of a cation at least divalent to the matrix obtained in step b),
d) optionally carrying out the lyophilization of the matrix obtained in step c).

In one embodiment, in step c), the solution of a soluble salt of a cation at least divalent is a divalent-cation solution.

In one embodiment, the soluble divalent-cation salts are calcium salts, the counterion of which is selected from the chloride, the D-gluconate, the oxalate, the hydroxide, the formate, the D-saccharate, the phosphate, the carbonate, the acetate, the sulphate, the L-ascorbate, the L-tartrate, the L-lactate, the glutamate or the aspartate.

In one embodiment, the soluble divalent-cation salt is calcium chloride.

The invention is illustrated by the following examples.

Synthesis of Carboxymethyl Dextran Functionalized with the Sodium Salt of Tryptophan (Polymer 1)

Step 1: Carboxymethyl Dextran Modified with the Ethyl Ester of Tryptophan

This amphiphilic polymer is synthesized starting from a carboxymethyl dextran having a degree of carboxymethyl substitution per saccharidic unit of 1.0 and an average molar mass of 60 kg/mol. The ethyl ester of tryptophan is grafted onto the carboxylic acids of this polymer according to a conventional coupling method in organic solvent, using ethyl chloroformate and N-methylmorpholine. After diluting the reaction medium in water and adjusting the pH to 7 by adding 1N NaOH, the polymer is purified by ultrafiltration. The final polymer is characterized by:
a degree of TrpOEt substitution per saccharidic unit of 0.45, determined by ¹H NMR in D₂O/NaOD;
a degree of carboxylate (methylcarboxylate) substitution of 0.55, determined by a potentiometric assay.
   Step 2: Carboxymethyl Dextran Modified with the Sodium Salt of Tryptophan, Polymer 1

This amphiphilic polymer is obtained by basic hydrolysis of the carboxymethyl dextran modified with the ethyl ester of tryptophan. 1N sodium hydroxide (3.79 ml) is added to an aqueous solution of the carboxymethyl dextran modified with the ethyl ester of tryptophan (64 ml at 31 mg/ml) so as to reach pH 12.7. The solution obtained is stirred overnight at ambient temperature. The polymer is purified by dialysis against water (0.9% NaCl and H₂O). The final polymer is characterized by:
a degree of TrpONa substitution per saccharidic unit of 0.45, determined by ¹H NMR in D₂O/NaOD;
a degree of carboxylate (methylcarboxylate, tryptophan carboxylate) substitution of 1.0, determined by a potentiometric assay.

### EXAMPLE 1

### Preparation of the Collagen Sponge/rhBMP-2 Implant

Implant 1: 40 μl of a solution of rhBMP-2 at 0.5 mg/ml are introduced sterilely into a Helistat type sterile 200 mm³ crosslinked collagen sponge (Integra LifeSciences, Plainsboro, N.J.). The solution is left to incubate for 30 minutes in the collagen sponge before use. The dose of BMP-2 is 20 μg.

### EXAMPLE 2

### Preparation of the Collagen Sponge/rhBMP-2/PDGF-BB Implant

Implant 2: 20 μl of a solution of rhBMP-2 at 1.0 mg/ml and also 20 μl of a solution of rhPDGF-BB at 1.0 mg/ml are introduced sterilely into a Helistat type sterile 200 mm³ crosslinked collagen sponge (Integra LifeSciences, Plainsboro, N.J.). The solution is left to incubate for 30 minutes in the collagen sponge before use. The dose of BMP-2 and also that of PDGF-BB are each 20 μg.

### EXAMPLE 3

### Preparation of the rhBMP-2/Polymer 1 Complex

Formulation 1: 50 μl of a solution of rhBMP-2 at 1.5 mg/ml are mixed with 100 μl of a solution of polymer 1 at 37.5 mg/ml. The solutions of rhBMP-2 and of polymer 1 are buffered at pH 7.4. This solution is left to incubate for two hours at 4° C. and filtered sterilely through 0.22 μm.

Formulation 2: 50 μl of a solution of rhBMP-2 at 0.75 mg/ml are mixed with 100 μl of a solution of polymer 1 at 37.5 mg/ml. The solutions of rhBMP-2 and of polymer 1 are buffered at pH 7.4. This solution is left to incubate for two hours at 4° C. and filtered steriley through 0.22 μm.

### EXAMPLE 4

### Preparation of the rhPDGF-Bb/Polymer 1 Complex

Formulation 3: 50 μl of a solution of rhPDGF-BB at 1.5 mg/ml are mixed with 100 μl of a solution of polymer 1 at 37.5 mg/ml. The solutions of rhPDGF-BB and of polymer 1 are buffered at pH 7.4. This solution is left to incubate for two hours at 4° C. and filtered steriley through 0.22 μm.

### EXAMPLE 5

### Preparation of the Implants of Collagen Sponge/BMP-2/Polymer 1 Complex in the Presence of Calcium Chloride, which are Lyophilized

Implant 3: 40 μl of formulation 1 are introduced into a Helistat type sterile 200 mm³ crosslinked collagen sponge (Integra LifeSciences, Plainsboro, N.J.). The solution is left to incubate for 30 minutes in the collagen sponge before adding 100 μl of a solution of calcium chloride at a concentration of 18.3 mg/ml. The sponge is then frozen and lyophilized sterilely. The dose of BMP-2 is 20 μg.

### EXAMPLE 6

### Preparation of the Implants of Collagen Sponge/BMP-2/PDGF-BB/Polymer 1 Complex in the Presence of Calcium Chloride, which are Lyophilized

Implant 4: 20 μl of formulation 2 and also 20 μl of formulation 3 are introduced into a Helistat type sterile 200 mm³ crosslinked collagen sponge (Integra LifeSciences, Plainsboro, N.J.). The solution is left to incubate for 30 minutes in the collagen sponge before adding 100 μl of a solution of calcium chloride at a concentration of 18.3 mg/ml. The sponge is then subsequently frozen and lyophilized sterilely. The dose of BMP-2 is 5 μg and that of PDGF-BB is 10 μg.

### EXAMPLE 7

### Evaluation of the Osteoinductive Capacity of the Various Formulations

The objective of this study is to demonstrate the osteoinductive capacity of the various formulations in a model of ectopic bone formation in the rat. Male rats weighing 150 to 250 g (Sprague Dawley OFA-SD, Charles River Laboratories France, B.P. 109, 69592 I'Arbresle) are used for this study.

An analgesic treatment (buprenorphine, Temgesic®, Pfizer, France) is administered before the surgical procedure. The rats are anaesthetized by inhalation of an O₂-isoflurane mixture (1-4%). The fur is removed by shaving over a wide dorsal area. The skin of this dorsal area is disinfected with a solution of povidone-iodine (Vetedine® solution, Vetoquinol, France).

Paravertebral incisions of approximately 1 cm are made in order to free the right and left dorsal paravertebral muscles. Access to the muscles is made by transfascial incision. Each of the implants is placed in a pocket in such a way that no compression can be exerted thereon. Four implants are implanted per rat (two implants per site). The implant opening is then sutured using a polypropylene thread (Prolene 4/0, Ethicon, France). The skin is re-closed using a nonabsorbable suture. The rats are then returned to their respective cages and kept under observation during their recovery.

At 21 days, the animals are anaesthetized with an injection of tiletamine-zolazepam (Zoletil® 25-50 mg/kg, 1M, VIRBAC, France).

The animals are then sacrificed by euthanasia, by injecting a dose of pentobarbital (Dolethal®, VETOQUINOL, France). A macroscopic observation of each site is then carried out; any sign of local intolerance (inflammation, necrosis, haemorrhage) and the presence of bone and/or cartilage tissue are recorded and graded according to the following scale: 0: absence, 1: weak, 2: moderate, 3: marked, 4: substantial.

Each of the implants is removed from its implantation site and macroscopic photographs are taken. The size and the weight of the implants are then determined. Each implant is then stored in a buffered 10% formol solution.

Results:

This in vivo experiment makes it possible to measure the osteoinductive effect of BMP-2 by placing the implant in a muscle on the back of a rat. This non-bone site is termed ectopic.

The macroscopic observations of the explants enable us to evaluate the presence of bone tissues and the mass of the implants.

| | rhBMP-2 | rhPDGF-BB | Presence of | Mass of |
|---|---|---|---|---|
| Implant | (μg) | (μg) | bone tissues | implants (mg) |
| Implant 1 | 20 | — | 3.6 | 37 |
| Implant 2 | 20 | 20 | 3.8 | 68 |
| Implant 3 | 20 | — | 3.8 | 267 |
| Implant 4 | 5 | 10 | 3.8 | 401 |

A dose of 20 μg of BMP-2 in a collagen sponge (Implant 1) makes it possible to obtain ossified implants with an average weight of 37 mg, after 21 days.

For the same dose of BMP-2 combined with 20 μg of PDGF-BB, the mass of the implants almost doubled since the implants weigh, on average, 68 mg. The PDGF-BB therefore clearly stimulates the osteogenic activity of the BMP-2 and a synergic activity is observed.

This observation is even more marked in the presence of a formulation based on complexes of BMP-2 and PDGF-BB and calcium chloride. An implant containing 20 μg of BMP-2 combined with 1 mg of polymer 1 results in bones having an average mass of 267 mg. When the implant contains only 5 μg of BMP-2, and 10 μg of PDGF-BB, which are combined with 1 mg of polymer 1, the mass of the explanted bones is much higher, on average 401 mg.

1. Osteogenic synergic composition comprising at least one osteogenic growth factor, and at least one growth factor having a chemoattractant and angiogenic capacity.

2. Composition according to claim 1, wherein the osteogenic growth factor is selected from the group of therapeutically active BMPs (Bone Morphogenetic Proteins).

3. Composition according to claim 2, wherein the osteogenic proteins are selected from the group constituted of BMP-2 (dibotermin-alfa), BMP-4, BMP-7 (eptotermin-alfa), BMP-14 and GDF-5, alone or in combination.

4. Composition according to claim 1, wherein the chemoattractant and angiogenic growth factor is a protein selected from the group constituted of PDGF, VEGF or FGF, alone or in combination.

5. Osteogenic synergic composition according claim 1, wherein the chemoattractant and angiogenic growth factor is PDGF.

6. Synergic composition according to claim 3, wherein the chemoattractant and angiogenic growth factor is PDGF.

7. Synergic composition according to claim 6, comprising at least BMP-2 and PDGF-BB.

8. Synergic composition according to claim 6, comprising at least BMP-7 and PDGF-BB.

9. Composition according to claim 1, wherein it further comprises an anionic polysaccharide selected from the group constituted of anionic polysaccharides functionalized with hydrophobic derivatives selected from the derivatives of functionalized dextrans bearing hydrophobic substituents such as tryptophan and tryptophan derivatives.

10. Composition according to claim 9, wherein the functionalized dextran corresponds to the following general formula: 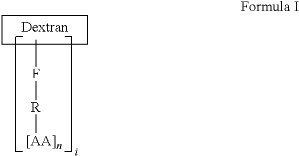
R being a chain containing between 1 and 18 carbons, which is optionally branched and/or unsaturated, which comprises one or more heteroatoms, such as O, N and/or S, and which has at least one acid function;
F being either an ester, a thioester, an amide, a carbonate, a carbamate, an ether, a thioether, or an amine;
AA being a hydrophobic L- or D-amino acid residue produced from the coupling between the amine of the amino acid and an acid borne by the R group, said hydrophobic amino acid being selected from tryptophan derivatives, such as tryptophan, tryptophanol, tryptophanamide, 2-indole ethylamine, and alkali-metal cation salts thereof;
i is the molar fraction of substituent F—R—[AA]ₙ per glycosidic unit and is between 0.1 and 2;
n is the molar fraction of AA-substituted groups and is between 0.05 and 1;
when R is not substituted with AA, then the acid(s) of the R group is (are) cation carboxylates, the cation preferably being a cation of an alkali metal such as Na or K,
said dextran being amphiphilic at neutral pH.

11. Composition according to claim 1, wherein it further comprises a soluble salt of a divalent cation selected from the group constituted of calcium, magnesium or zinc cations.

12. Composition according to claim 11, wherein the soluble divalent-cation salt is a calcium salt, the counterion of which is selected from the chloride, the D-gluconate, the oxalate, the hydroxide, the formate, the D-saccharate, the phosphate, the carbonate, the acetate, the sulphate, the L-ascorbate, the L-tartrate, the L-lactate, the glutamate or the aspartate.

13. Composition according to claim 12, wherein the soluble divalent-cation salt is calcium chloride.

14. Composition according to claim 1, wherein it further comprises a matrix selected from matrices based on natural collagen.

15. Composition according to claim 1, wherein it further comprises multivalent cations selected from the group constituted of iron cations, aluminium cations and cationic polymers selected from polylysine, spermine, protamine and fibrin, alone or in combination.

16. Composition according to claim 1, wherein it is in the form of a lyophilisate.
